(19) United States
(12) Patent Application Publication
(10) Pub. No.: US 2018/0158201 A1
(43) Pub. Date: Jun. 7, 2018

(54) APPARATUS AND METHOD FOR REGISTERING PRE-OPERATIVE IMAGE DATA WITH INTRA-OPERATIVE LAPAROSCOPIC ULTRASOUND IMAGES

(71) Applicant: UCL Business PLC, London (GB)

(72) Inventors: Stephen Thompson, London (GB); Matt Clarkson, London (GB); David Hawkes, London (GB); Yi Song, London (GB)

(73) Assignee: UCL Business PLC, London (GB)

(21) Appl. No.: 15/568,413

(22) PCT Filed: Jun. 17, 2016
(86) PCT No.: PCT/GB2016/051818

§ 371 Date: Oct. 20, 2017

(30) Foreign Application Priority Data
Apr. 22, 2015 (GB) 1506842.2

(51) Int. Cl. G06T 7/33; G06T 7/11; G06T 7/73; A61B 8/12

(52) U.S. Cl. CPC G06T 7/344; G06T 7/11; G06T 7/74; A61B 8/12; G06T 2207/10132; G06T 2200/08; G06T 2207/10081; G06T 2207/30056; G06T 2207/30101; G06T 2207/10016; G06T 2200/04; G06T 2207/10088

(57) ABSTRACT

A method and apparatus are provided for registering pre-operative three dimensional (3-D) image data of a deformable organ comprising vessels with multiple intra-operative two-dimensional (2-D) ultrasound images of the deformable organ acquired by a laparoscopic ultrasound probe during a laparoscopic procedure. The apparatus is configured to: generate a 3-D vessel graph from the 3-D pre-operative image data; use the multiple 2-D ultrasound images to identify 3-D vessel locations in the deformable organ; determine a rigid registration between the 3-D vessel graph from the 3-D pre-operative image data and the identified 3-D vessel locations in the deformable organ; and apply said rigid registration to align the pre-operative three dimensional (3-D) image data with the two-dimensional (2-D) ultrasound images, wherein the rigid registration is locally valid in the region of the deformable organ of interest for the laparoscopic procedure.

### FIELD

The present invention relates to a method and apparatus for registering pre-operative three dimensional (3-D) image data of a deformable organ comprising vessels with multiple intra-operative two-dimensional (2-D) ultrasound images of the deformable organ acquired by a laparoscopic ultrasound probe.

### BACKGROUND

In the UK, approximately 1800 liver resections are performed annually for primary or metastatic cancer. Liver cancer is a major global health problem, and 150,000 patients per year could benefit from liver resection. Currently, approximately 10% of patients are considered suitable for laparoscopic liver resection, mainly those with small cancers on the periphery of the liver. Potentially, laparoscopic resection has significant benefits in reduced pain and cost savings due to shorter hospital stays [7]. Such laparoscopic surgery is regarded as minimally invasive, in that equipment or tools for performing the procedure are inserted into the body relatively far from the surgical site and manipulated through trocars. However, larger lesions and those close to major vascular and/or biliary structures are generally considered high risk for the laparoscopic approach, mainly due to the restricted field of view and lack of haptic feedback.

In many clinical procedures, pre-operative 3-dimensional images are acquired using a modality such as X-ray computed tomography (CT) or magnetic resonance imaging (MRI). However, CT/MRI imaging is generally not feasible in an intra-operative context, where ultrasound (US) is generally used (for reasons such as safety and convenience). However, certain items of clinical interest, e.g. cancers/tumours, are relatively difficult to see in an US image, and also the US image quality (e.g. signal-to-noise ratio) may be relatively low compared to the pre-operative CT/MRI imaging, in part because the acquisition of the former has to fit in with the particular constraints of being performed in an intra-operative context.

This leads to the situation in which it is desirable, in an intra-operative context, to register a newly acquired US image against a pre-operative CT/MRI image, in order to allow the US image to be displayed in positional correspondence with (e.g. overlaid upon) the earlier CT/MRI image. This then allows a surgeon (for example) to track the position of a surgical instrument (as visible in the US image) in relation to a desired tumour or other biological feature (as visible in the CT/MRI image). However, this image registration between the US and CT/MRI images is complicated by the fact that many anatomical features are non-rigid, and hence prone to deformation or changes in shape, for example, due to changes in posture of the subject, and/or the surgical intervention itself.

Previously reported commercial systems have performed such image registration, for example using surfaces of a liver reconstructed by using a dragged pointer [14,] or manual identification of four points (CAS-ONE 1—http://www.cascination.com). The former approach is prone to error due to direct contact with a soft tissue, while both are limited to a global rigid registration which is clearly unrealistic given the abdominal insufflation needed in laparoscopy. A previously developed system [24] for laparoscopic guidance is based on dense stereo surface reconstruction [25] and then using an iterative closest point (ICP) [5] algorithm for alignment to a surface derived from a preoperative CT model. However, the research literature suggests that deformable registration is highly preferable for image guidance [13, 23] in at least some clinical situations. On the other hand, deformable models are difficult to validate [19] and may have multiple plausible solutions. It is also more difficult for a surgeon who is performing an operation to understand the registration accuracy of such a deformable image registration.

In the literature, Aylward proposed a rigid body registration of 3D B-mode ultrasound to preoperative CT for radio frequency ablation, based on a feature-to-image metric [2]. Lange, however, used a feature-to-feature method by extracting vessel centre lines from CT data and 3D power Doppler ultrasound and then used ICP followed by multi-level B-splines for non-rigid alignment [15]. This was subsequently extended to incorporate vessel branch points as registration constraints [16]. The branch points were automatically identified in advance of surgery in the CT data, but selected manually in the ultrasound.

Accurate segmentation (identification of different anatomical features, especially in 3-D data sets) is a critical prerequisite for feature-based registration, and ultrasound image segmentation is a particularly challenging problem due to the relatively low signal-to-noise ratio, see the review of Noble [20]. Subsequently, Guerrero used an ellipse model to constrain an edge detection algorithm [12], thereby extracting vessels from ultrasound data for assessment of deep vein thrombosis. Later, Schneider used power Doppler ultrasound to initialise and guide vessel segmentation in B-mode images [22], replacing the previously required [12] manual initialisation of vessel centres. A scale-space blob detection approach has been used by Dagon et al. [8] and Anderegg et al. [1] to initialise vessel regions and approximate vessel walls using an ellipse model.

An alternative approach to feature-to-feature registration is image-to-image registration. Penney et al. [21] transformed a sparse set of freehand ultrasound slices to probability maps and registered with resampled and pre-processed CT data. Subsequently, Wein et al. [26] used a magnetic tracker to perform freehand 3D ultrasound registration of a sweep of data to pre-processed CT images using a semi-affine (rotations, translations, 2 scaling, 1 skew) transformation. This work was extended to non-rigid deformation using B-splines and tested in a neurosurgical application [27].

However, there still exist challenges that are specific to the use of freehand laparoscopic ultrasound (LUS) in surgical applications. For example, the methods of Aylward et al. [2] and Lange et al. [16], as discussed above, are based on a 3D percutaneous probe. The probe is held stationary while a mechanical motor sweeps the ultrasound transducer in a predictable arc. Unfortunately, there are currently no commercially available laparoscopic 3D ultrasound probes. Wein's work is based on a percutaneous probe which is swept through a volume to collect a dense set of ultrasound image slices [26 (which can then be assembled into a 3-D data set)], while Penney's work collects a sparse set of ultrasound image slices [21]. However, in a freehand laparoscopic setting, port positions and positioning of the LUS probe are often restrictive, and control of the motion during a sweep of data is often difficult, resulting in jerky motion. Moreover, the relatively small field of view makes the context difficult to interpret, and it can be difficult to obtain elliptical vessel outlines.

### SUMMARY

The invention is defined in the appended claims.

A method and apparatus are provided for registering pre-operative three dimensional (3-D) image data of a deformable organ comprising vessels with multiple intra-operative two-dimensional (2-D) ultrasound images of the deformable organ (such as the liver) acquired by a laparoscopic ultrasound probe during a laparoscopic procedure. The apparatus is configured to: generate a 3-D vessel graph from the 3-D pre-operative image data; use the multiple 2-D ultrasound images to identify 3-D vessel locations in the deformable organ; determine a rigid registration between the 3-D vessel graph from the 3-D pre-operative image data and the identified 3-D vessel locations in the deformable organ; and apply said rigid registration to align the pre-operative three dimensional (3-D) image data with the two-dimensional (2-D) ultrasound images, wherein the rigid registration is locally valid in the region of the deformable organ of interest for the laparoscopic procedure.

The approach described herein is based on a locally rigid registration. It is shown by experiment that this registration may be sufficiently accurate for surgical guidance for a laparoscopic procedure, even in respect of a deformable organ such as the liver, having regard to the fact that laparoscopic procedures tend to involve rather constrained (local) spatial regions.

Typically the pre-operative three dimensional (3-D) image data comprises magnetic resonance (MR) or computed tomography (CT) image data, while the multiple intra-operative two-dimensional (2-D) ultrasound images comprise 2D ultrasound slices at different orientations and positions through the region of the deformable organ of interest for the laparoscopic procedure. The laparoscopic ultrasound probe may include a tracker to provide tracking information for the probe that allows the 2D ultrasound slices at different orientations and positions to be mapped into a consistent 3-D space.

In some implementations, generating a 3-D vessel graph from the 3-D pre-operative image data comprises: segmenting the 3-D pre-operative image data into anatomical features including the vessels; and identifying the centre-lines of the segmented vessels to generate the 3-D vessel graph. Using the multiple 2-D ultrasound images to identify 3-D vessel locations in the deformable organ comprises: identifying the locations of vessels within individual 2-D ultrasound images; and converting the identified locations of vessels within an individual 2-D ultrasound image into corresponding 3-D locations of vessels using tracking information for the laparoscopic ultrasound probe. Identifying the locations of vessels within an individual 2-D ultrasound images may comprises applying a vessel enhancement filter to the individual ultrasound image; thresholding the filtered image; and fitting ellipses to the thresholded image, whereby a fitted ellipse corresponds to a cross-section through a vessel in the individual ultrasound image.

Typically, determining the rigid registration between the 3-D vessel graph and the identified 3-D vessel locations in the deformable organ includes determining an initial alignment based on two or more corresponding anatomical landmarks in the 3-D vessel graph from the pre-operative image data and the identified 3-D vessel locations from the intra-operative ultrasound images. The initial alignment may be performed by manually identifying the corresponding anatomical landmarks, but in some cases an automatic identification may be feasible. The anatomical landmarks may comprise vessel bifurcations or any other suitable features.

Determining the rigid registration may include determining an alignment between the 3-D vessel graph from the pre-operative image data and points representing the identified 3-D vessel locations from the intra-operative ultrasound images using an iterative closest points algorithm (other algorithms are also available for performed such a registration). The identified 3-D vessel locations may comprise a cloud of points in 3D space, each point representing the centre-point of a vessel, wherein the vessel graph comprises the centre-lines of the vessels identified in the pre-operative image data, and wherein the rigid registration is determined between the vessel graph of centre-lines and the cloud of points. The rigid registration (however determined) can then be used to align the pre-operative three dimensional (3-D) image data with the two-dimensional (2-D) ultrasound images. Note that this alignment with the US images may be applied with respect to the raw MR/CT images, or to image data derived from the raw images (such as a segmented model).

A real-time, intra-operative, display of the pre-operative three dimensional (3-D) image data registered with the two-dimensional (2-D) ultrasound images may be provided. The laparascopic ultrasound probe may includes a video camera, and the method may further comprise displaying a video image from the video camera in alignment with the three dimensional (3-D) image data and the two-dimensional (2-D) ultrasound images.

The above approach helps to provides a wider spatial context and greater accuracy by aligning data obtained pre-operatively and derived from magnetic MR or CT scans with US images in a laparoscopic procedure. For example, a freehand laparoscopic ultrasound (LUS)-based system is provided that registers liver vessels in ultrasound (US) with MR/CT data.

### BRIEF DESCRIPTION OF THE DRAWINGS

The invention is now described by way of example only with reference to the following drawings in which:

FIG. 1 schematically represents an overview of the registration process in accordance with some implementations of the invention.

FIG. 2 shows an example of applying the registration transformation to anatomical models derived from preoperative CT data in accordance with some implementations of the invention.

FIG. 3 shows and example of vessel segmentation on CT data in accordance with some implementations of the invention.

FIG. 4 illustrates the creation of a Dip image in accordance with some implementations of the invention.

FIG. 5 illustrates of outlier rejection for a vessel in accordance with some implementations of the invention.

FIG. 6 shows an example of corresponding landmarks and vectors in the hepatic vein, as used for initial alignment for the registration procedure in accordance with some implementations of the invention.

FIG. 7 illustrates an evaluation of ultrasound calibration described herein using an eight-point phantom.

FIG. 8 illustrates a validation of the vessel segmentation described herein.

FIG. 9 illustrates a validation of the vessel registration described herein on the phantom of FIG. 8.

FIG. 10 illustrates hepatic vein landmark positions used for the measuring target registration error (TRE) in the registration procedure described herein.

FIG. 11 shows an evaluation of registration accuracy with locally rigid registration as described herein.

FIG. 12 shows an evaluation of navigation accuracy with locally rigid registration as described herein. The errors are shown as a function of distance from the reference landmarks.

### DETAILED DESCRIPTION

Aspects and features of certain examples and embodiments of the present invention are described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional technique for implementing such aspects and features.

Described herein is a locally rigid registration system to align pre-operative MR/CT image data with intra-operative ultrasound data acquired using a 2D laparoscopic ultrasound (LUS) probe during a laparoscopic procedure, such as laparoscopic resection of the liver. Such CT or MR image data usually encompasses the entire organ, but may in some cases only represent a part of the organ.

As described in more detail below, some implementations of the above approach extract vessel centre lines from preoperative MR/CT image data (relating to a soft, deformable organ such as the liver) in a similar manner to [1, 8, 22]. Features, such as bifurcation points where a vessel splits into two vessels can be identified, either manually or automatically, from the vessel centre lines and used as landmarks for performing registration. In addition, a series of 2D ultrasound images of a local region of the soft deformable organ are obtained intra-operatively using a 2D LUS probe. In this regard, the 2D LUS probe is scanned (freehand) over a part of the soft deforming organ of interest for the laparoscopic procedure to obtain a sequence of images representing slices through the local region of the organ at different positions and locations. The 2D LUS probe is typically a 2D array of transducers positioned along the length of a laparoscope and configured to receive reflected US.

From the sequence of 2D ultrasound images, vessel centre-points (i.e., the centres of vessels identified in the images) are obtained, for example, by fitting an ellipse to contours of the identified vessels and, providing the ellipse satisfies certain criteria, the centre of the fitted ellipse then becomes the vessel centre-point. Vessel centre-points can be determined as appropriate for each 2D US image.

In some implementations, the 2D laparoscopic probe is tracked using an electromagnetic (EM) tracker. The EM tracker allows external detectors to determine the (6-axis) position and orientation of the ultrasound probe, thereby enabling images obtained by the probe to be located within a consistent reference frame. The reference frame may (for example) be defined with reference to the frame of the operating theatre, or any other suitable frame. In addition, other methods for tracking the position of the US probe are known in the art.

Using the tracking information associated with each US image and the calibration of the 2D LUS probe itself (in terms of linear scale), the identified vessel centre-points can be given a three-dimensional co-ordinate in the reference frame. Thus a map of 3D vessel centre points can be created.

In some implementation, two or more anatomical landmarks are identified in the extracted vessel centre-lines from the pre-operative data and the corresponding landmarks are respectively identified in the derived vessel centre-points. These landmarks (and their correspondence with one another) may be identified manually. Using these landmarks, a first rigid registration of the pre-operative CT or MR image data to the 3D vessel centre points of the local region can be performed. This initial registration may, if desired, be refined by using a further alignment procedure, such as the iterative closest point registration procedure as described in [15, 22], which minimises the spatial distances between the vessel centre-lines and the vessel centre-points. In this way, the CT or MR image data can be aligned into the same reference frame as the ultrasound images.

This alignment is performed using a rigid registration, which is appropriate for transforming a rigid body from one reference frame to another. In particular, this rigid registration may involve translation, linear scaling and rotation, but (generally) not skew, or any non-linear transformations. For a rigid transformation, the relative locations of points within the transformed image therefore remain essentially constant.

It is noted that a deformable organ may change shape due to numerous factors, such as patient posture, the insertion of a medical instrument, patient breathing, etc. If two images of the deformable organ are acquired at different times, then it is more common to try to perform a non-rigid registration between such images, in order to allow for potential (and often expected) differences in deformation between the two images. However, non-rigid registration is complex and non-linear—consequently, it can be difficult to provide fully reliable results (e.g. where similar pairs of images produce similar registrations) and likewise difficult to assess maximum errors. This uncertainty makes clinical staff reluctant to use such non-rigid registration in an intra-operative environment.

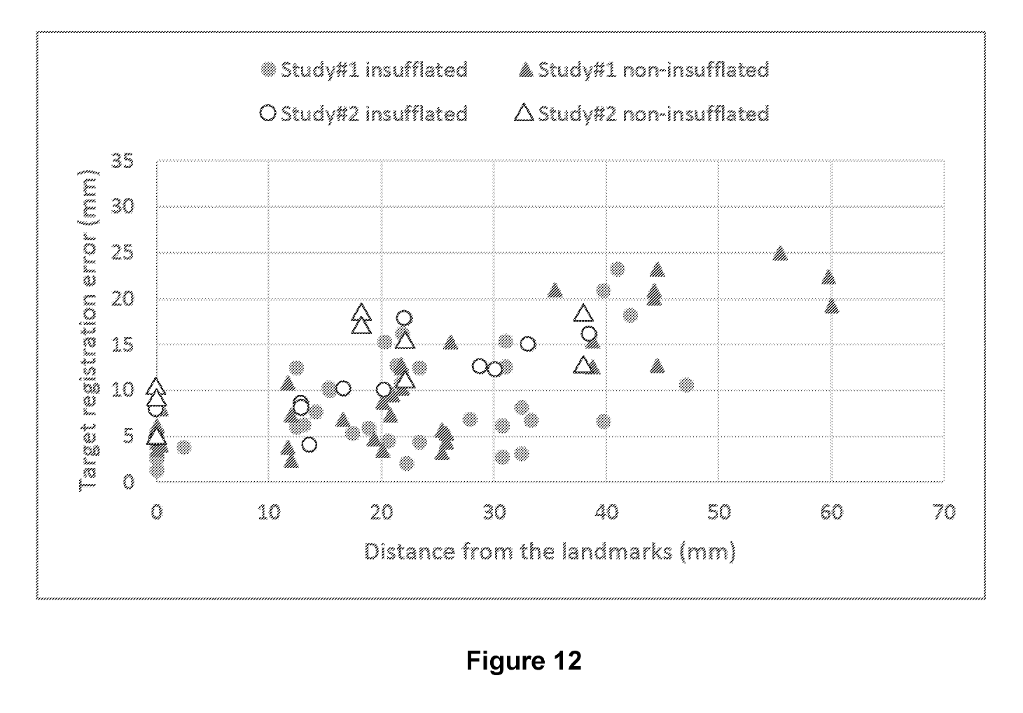

The approach described here performs a “local” rigid registration to a deformable organ. In other words, mathematically, the registration is a rigid registration, and so avoids the above issues with a non-rigid registration. Furthermore, this local rigid registration is utilised in a laparoscopic procedure, which is typically focussed on a relatively limited region of an organ. As shown below, within this (local) region, the rigid registration is sufficiently accurate for clinical purposes (at least according to the experiments performed below), even though it is recognised that larger registration errors will exist outside this region. In other words, the rigid registration itself is not “local” from a mathematical perspective, rather, the use and validity of the rigid registration is regarded as local to the region of interest and the image data used to determine the registration. As described in more detail below with respect to FIGS. 11 and 12 below, the accuracy of the registration declines as one moves further away from the local region, but the registration may remain accurate enough in the local region itself to provide reliable guidance for a clinician.

The registration process allows the CT or MR image data to be displayed in positional alignment with the intra-operative 2D US images. Such a display may adopt a side-by-side presentation, or may superimpose one image over the other. In addition, the laparoscope other provides a visual (video) view of the organ itself, and this visual view can also be present in conjunction with the pre-operative image data (in essence using the same registration as determined for the ultrasound, since the ultrasound and video data are both captured by the laparoscope and therefore share a common frame).

Although globally rigid [22] and additionally deformable [1, 8] registration of vessel models from CT and US data have been proposed, the present approach provides a registration procedure for a clinically usable laparoscopic ultrasound system based on a local rigid registration for use in a limited region of interest. It has been found that this approach is sufficient for image guidance without deformable modelling, following a thorough evaluation of errors using a phantom and during porcine laparoscopic liver resection.

### Method

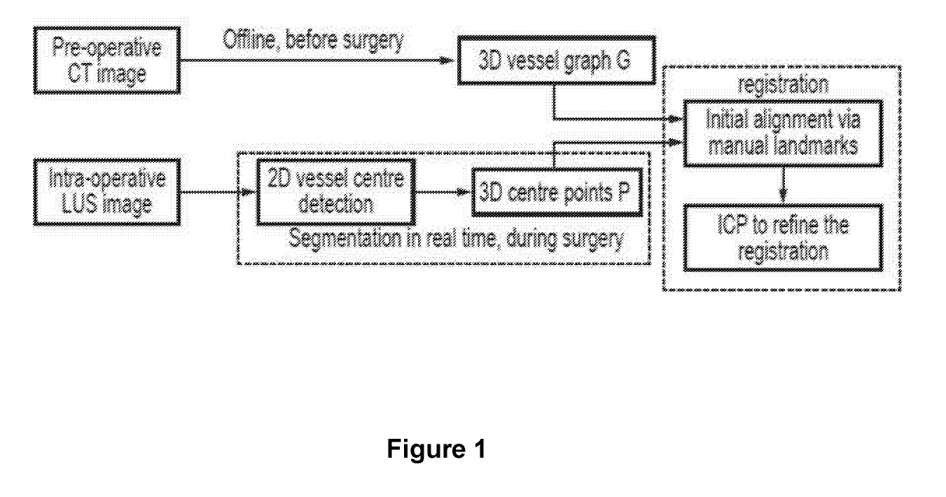

FIG. 1 shows an overview of the image registration process in accordance with some embodiments of the invention, in which vessel centre points P from ultrasound data are registered to a vessel centre-line graph G giving a rigid body transformation ^{G}T_{P}. In particular, in the method of FIG. 1, vessel centre points P are detected in 2D ultrasound images of an organ such as the liver which are acquired in real-time (intra-operatively). The 2D US images in effect represent slices at different orientations. The vessel centre points P are then converted into 3D space via an ultrasound calibration transformation and a tracking transformation. The pre-operative CT scan is pre-processed (before surgery) to extract a graph G representing vessel centre lines. The ultrasound-derived data P and CT-derived data G are then registered using manually picked landmarks and/or the ICP algorithm. The locally rigid registration transformation ^{G}T_{P} enables the pre-operative data to be visualised relative to the live ultrasound imaging plane.

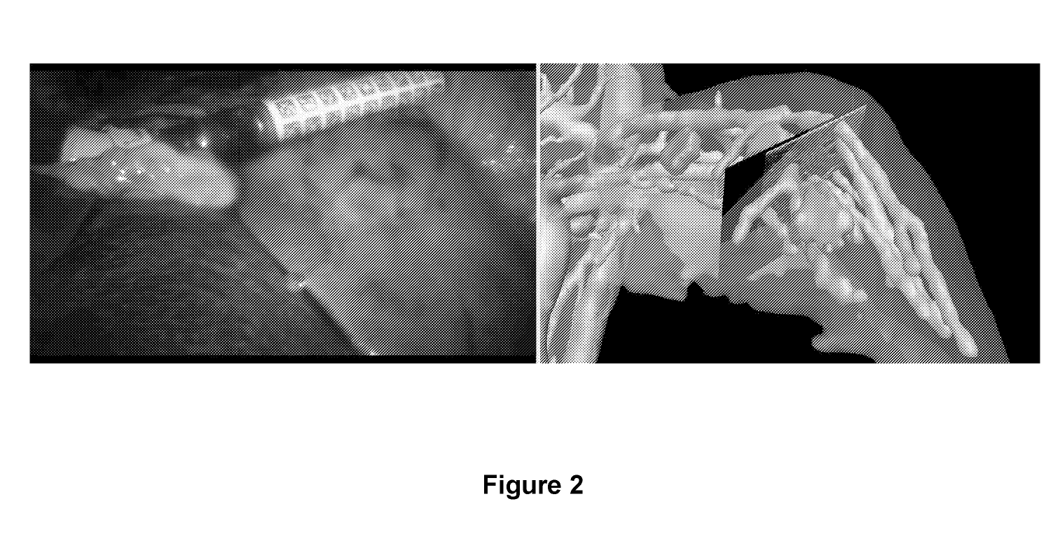

FIG. 2 shows an example of applying the registration transformation to an anatomical model derived from preoperative CT data to enable live visualisation of CT data, within the context of live ultrasound data (and laparoscopic video data). In particular, the left hand portion of FIG. 2 shows the laparoscopic video data, while the right-hand portion shows the CT data superimposed onto a live slice of 2-D ultrasound data.

### Pre-Processing Preoperative Data

A standard clinical tri-phase abdominal CT scan is obtained and segmented to represent one or more important structures such as the liver, tumours, arteries, hepatic vein, portal vein, gall bladder, etc. (See http://www.visiblepatient.com). Centre lines are then extracted from the CT scan using the Vascular Modelling Tool Kit (VMTK); further details about VMTK can be found at http://vmtk.org/tutorials/Centrelines.html. This yields a vessel graph G, which can be readily processed to identify vessel bifurcation points.

### Real-Time Ultrasound Segmentation

Previous work on 2D ultrasound vessel segmentation has generally used an ellipse model to constrain the edge detection process [1, 8, 12]. This approach assumes that vessels are imaged approximately perpendicular to the vessel centre line. However, this approval is often not practical for laparoscopic use, in which movement may be restricted by the position of a trocar. Moreover, it is unclear how this approach handles topological changes of the external contours of vessels in the 2D US images. Accordingly, the approach described herein utilises a flexible segmentation method that is not limited to cross-sectional scans, and can also cope with topology changes during the course of scanning.

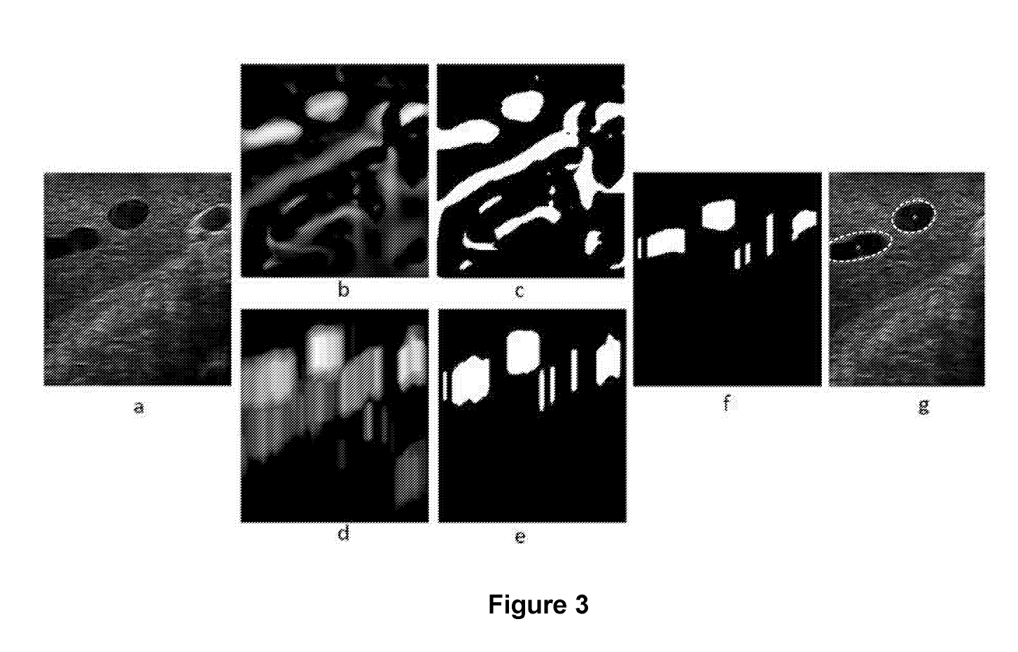

An example of the above segmentation is shown in FIG. 3. In particular FIG. 3a shows an ultrasound B-mode image; FIG. 3b shows a vessel enhanced image; FIG. 3c shows a thresholded vessel-enhanced image; FIG. 3d shows a Dip image generated using the approach described in [21]; FIG. 3e shows a thresholded Dip image; FIG. 3f shows the candidate seeds of vessels after the thresholded vessel-enhanced image is masked with the thresholded Dip image; and FIG. 3g shows vessel contours (depicted in red), fitted ellipses, and centre points (in green). These various stages of the processing of FIG. 3 will now be described in more detail.

### Vessel Enhancement Image

The standard B-mode ultrasound images have a low signal-to-noise ratio (FIG. 3a), so vessel structures are first enhanced for more reliable vessel segmentation. The multi-scale vessel enhancement filter from [10] is used, which is based on an eigenvalue analysis of the Hessian. The eigenvalues are ordered as |λ1|<|λ2|. The 2D “vesselness” of a pixel is measured by

$$\begin{matrix}{v}_{0} ( s ) = \{ \begin{matrix}0 & if {λ}_{2} < 0 \\ {e}^{( - \frac{{R}_{B}^{2}}{2 {β}^{2}} )} ( 1 - {e}^{( - \frac{{s}^{2}}{2 {c}^{2}} )} ) & \end{matrix} \\ where & ( 1 ) \\ {R}_{B} = {λ}_{1} / {λ}_{2} & ( 2 ) \\ S = \sqrt{{λ}_{1}^{2} + {λ}_{2}^{2}} & ( 3 )\end{matrix}$$

β=1 and c=10 are thresholds which control the sensitivity of the line filter to the measures R₈ and S (other values of these parameters can be used as appropriate).

In FIG. 3b, it can be seen that some common artefacts in the ultrasound images, e.g., shadows, are wrongly picked up by the enhancement filter. For many cases, using only the prior knowledge of the vessel intensity distributions is not sufficient to exclude those non-vessel regions. To improve robustness, the approach described herein adopts the Dip image as proposed by Penny et al. [21].

### Creation of the Dip Image

The Dip image (I_{dip}) was originally designed to produce vessel probability maps via a training data set. In the approach described herein, only the intensity differences (i.e., intensity dips) between regions of interest are used. The size of a region is determined by the diameter of vessels. No additional artefact removal step is required, except for a Gaussian filter over the US image. Since the experimental procedure described below targets the left liver lobe of a porcine liver for surgical guidance, the search range of vessel diameters is set from 9 to 3 mm (roughly equal to 100-40 pixels on the LUS image), as a porcine left lobe features relatively large vessels. However, it will be readily understood that different search ranges can be used as appropriate for different organs (and/or different species).

The Dip image is computed along the beam direction. As the experiment described below uses a linear 2D LUS probe, the beam directions can be modelled as image columns. A calculation is performed for three mean intensity values a, b and c, within regions [x+v/2, x+v], [x−v, x−v/2], [x−v/2, x+v/2], respectively, with x being a pixel at the ith column and v the vessel width. If c<b and c<a, every pixel in [x−v/2, x+v/2] on the Dip image will have the value bᵥ=min(a−c, b−c). This process is repeated for each v in [vₘᵢₙ, vₘₐₓ),]. The final pixel values at position [x−v/2, x+v/2] will be max(bᵥ). The steps above are repeated for every column of the US image and all pixels along that column. This can be parallelised easily as each column is processed independently of others. To reduce the search range of vessel diameters, a coarse-to-fine pyramidal approach may be used to speed up the process further.

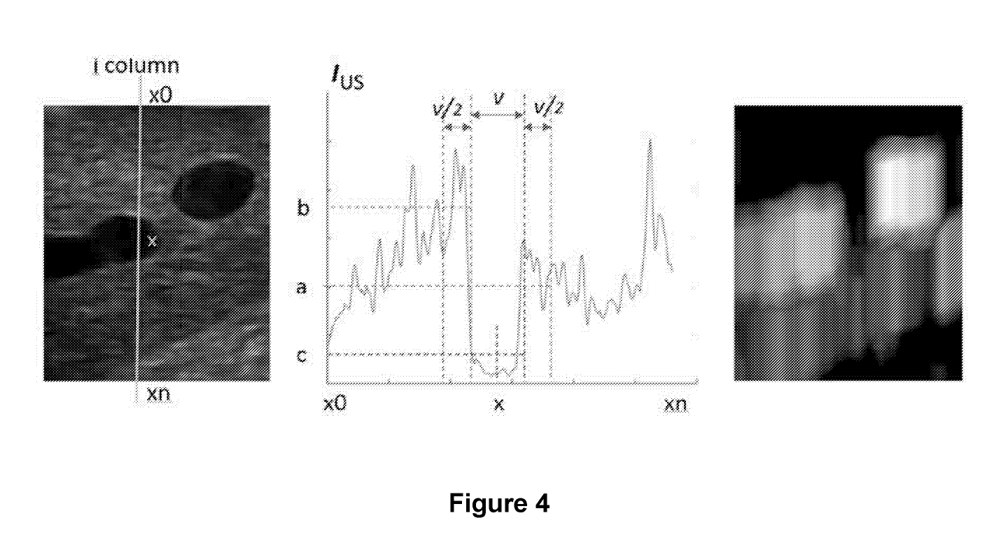

FIG. 4 depicts the creation of the Dip image. The image to the left represents the Gaussian blurred ultrasound image (I_{US}) (this is based on a portion of the image shown in FIG. 3a); the plot in the centre represents the intensity profile along line (x₀, xₙ) (as marked in the image to the left), wherein the location and size of image regions gives the values a, b and c; and the image to the right shows the resulting Dip image (this likewise corresponds to a portion of the image shown in FIG. 3f).

### Segmentation and Reconstruction

The vessel-enhanced image is thresholded at Tₑ to eliminate background noise; see FIG. 3c. In addition, a mask image (Iₘₐₛₖ) (see FIG. 3e) is created by applying a threshold (T_{d}) to the Dip image, this threshold may be set (for example) as half the maximum value of the Dip image. These two thresholds (Tₑ and T_{d}) are set having regard to the given B-mode ultrasound imaging parameters, e.g. gain, power, map, etc.

The de-noised vessel-enhanced image is then masked with Iₘₐₛₖ. Regions appearing on both images are kept, as shown in FIG. 3f. The intensity distribution of those regions can be further compared against the prior knowledge of vessel intensity and removed if they are not matching, i.e., they fall out of the vessel intensity range. The remaining pixels are candidate vessel seeds. The regions in the de-noised vessel enhancement image which contain such candidate seeds are identified as vessels and their contours are detected.

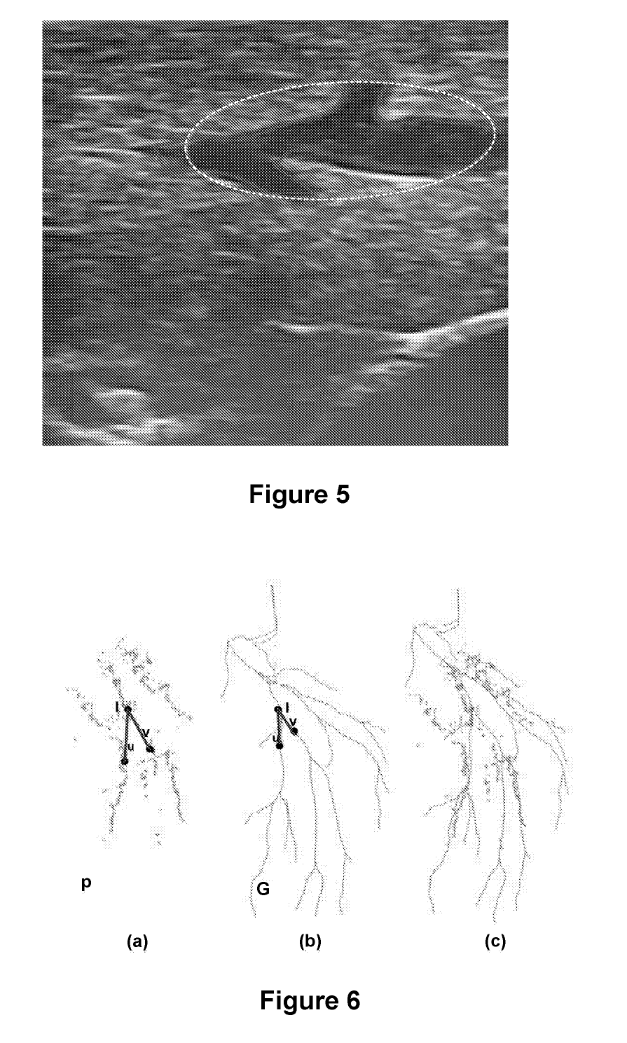

Since vessel centre points are employed for registration in the approach described herein, ellipses are fitted to those contours to derive centre points in each ultrasound image (as per FIG. 3g). Outliers can be excluded by defining minimal and maximal values for the (short axis) length of an ellipse and for the ratio of the axes of the ellipse. For example, when an image is scanned in a plane which is nearly parallel to a vessel centre-line direction, this results in large ellipse axes. Such an ellipse can be removed by constraining the short axis length to the pre-defined vessel diameter range [vₘᵢₙ, vₘₐₓ] as described in the above section “Creation of the Dip image” above. An additional criterion may be that the ratio of the axes should be larger than 0.5. Otherwise, the vessel may have been scanned in a direction less than 30° away from its centre-line direction, which often does not produce reliable ellipse centres. FIG. 5 shows an example of such outlier rejection, in which an ellipse has been fitted to the vessel outline, but the detected centre is rejected due to the ratio of the ellipse axes. After the vessel centres have been determined in 2D pixel coordinates, they are multiplied by the ultrasound calibration and the probe tracking transformation is applied to convert these 2D pixel coordinates into 3D data points (P), which can then be used to register the preoperative CT data to the patient in the operation room.

### Registration

For performing the image registration, a landmark L and two vectors, u and v are defined (identified) on the preoperative centre-line model G, along with their correspondences L′, u′, v′ in the derived centre points P. This initial correspondence may be determined manually (such as in the experiments described below), but might be automated instead. An initial rigid registration is therefore obtained by the alignment of landmarks {L, L′}, which gives the translation, and vectors {u, u′} and {v, v′}, which computes the rotation. After this initial alignment has been determined, the ICP algorithm [5] is applied to further refine the registration of the pre-operative data G to the intra-operative data P.

FIG. 6 shows an example having corresponding landmarks and vectors in the hepatic vein that are used for providing an alignment (registration) between the CT and US image data. In particular, FIG. 6a shows intra-operative centre points P obtained from intra-operative ultrasound images; FIG. 6b depicts pre-operative vessel centre-line model G obtained from the pre-operative image data, such as CT or MR image data; and FIG. 6c shows the pre-operative centre-line model G aligned to the intra-operative centre points P using an ICP algorithm as referenced above.

### Experiments and Results

Experiments were performed to determine the overall registration accuracy of the approach described herein and to identify sources of error from various component parts (see the sections “Ultrasound calibration error” and “Vessel segmentation error” below). The system provided for the experiments uses an electromagnetic (EM) tracker, which is known to display tracking inaccuracies due to magnetic field inhomogeneities [11]. Various works have tried to mitigate against such EM tracking inaccuracies by calibration [18] and combination with optical trackers [9]. The focus of this work concerns the practicalities of intra-operative registration, so the manufacturer-claimed position accuracy of 1.4 mm RMS and orientation accuracy of 0.5° RMS for the EM tracker are adopted herein.

A significant point for surgical navigation is that while the approach described herein determines the registration transformation ^{P}T_{G} from preoperative data G to intraoperative data P, the actual navigation accuracy is determined by the combination of the registration accuracy, the EM tracking accuracy as the probe moves, the US calibration accuracy and the deformation of the liver due to the US probe itself. For this reason, separate data are used to assess the registration accuracy (see the section below “Registration accuracy: in vivo”), and the navigation accuracy (see the section below: “Navigation accuracy: in vivo”).

The experiments described in these two sections utilised vessel models derived from CT scans taken using pneumoperitoneum (insufflated), which are not available clinically. Accordingly, in section below “Comparison of insufflated versus non-insufflated models”, the registration and navigation accuracy when registering to CT-derived vessel models are compared for with pneumoperitoneum (insufflated) and for without pneumoperitoneum (non-insufflated). The US images for these experiments were collected under controlled breathing (Boyles apparatus), as discussed later.

### Experimental Set-Up

The data acquisition system for handling the intra-operative US images is built upon the NifTK platform [6]. Live LUS images were acquired at 25 frames per second (fps) from an Analogic SonixMDP ultrasound machine (http://www.analogicultrasound.com) operated in combination with a Vermon (http://www.vermon.com) LP7 linear probe (for 2D US scanning). An Ascension (http://www.ascension-tech.com) 3D Guidance medSafe mid-range electromagnetic (EM) tracker was used to track the LUS probe at 60 fps via a six-degrees-of-freedom (6-DOF) sensor (Model 180) attached to the articulated tip.

### Ultrasound Calibration Error

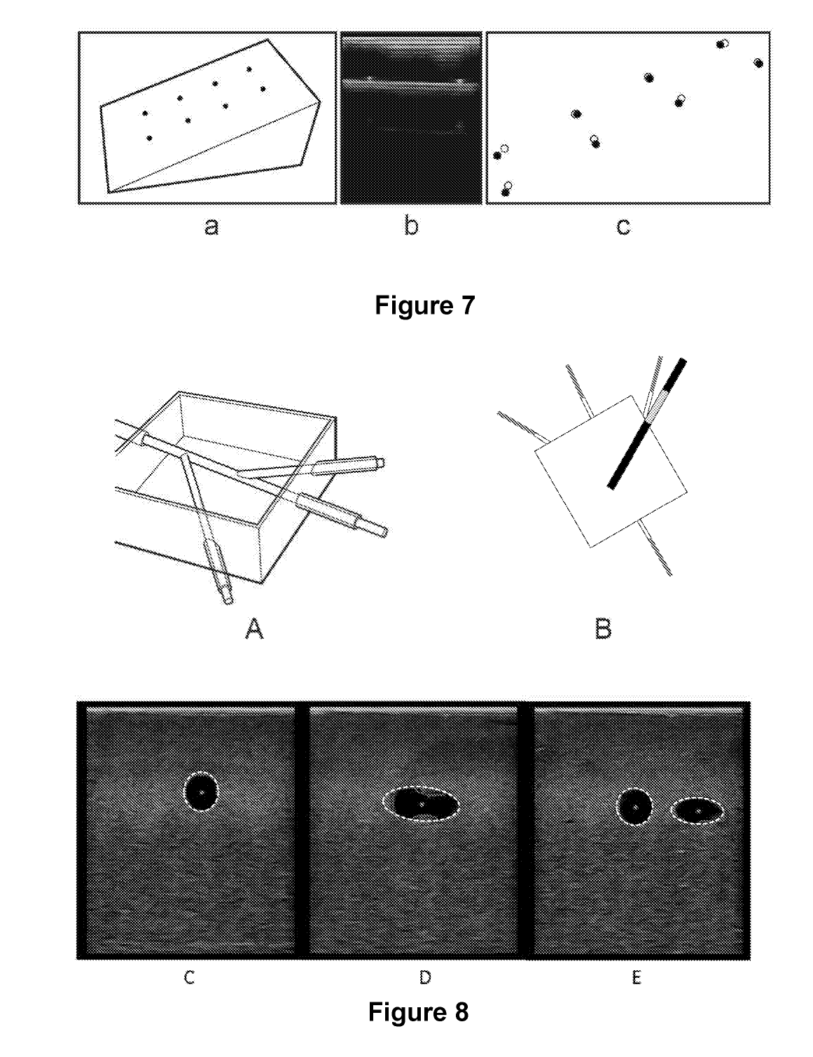

In this experiment, the LUS probe was calibrated at a scanning depth of 45 mm before surgery using an invariant point method as in [17]. The scanning depth of the LUS probe was not changed throughout the experiments. The validation phantom is shown in FIG. 7a, and described further in [4]. More particularly, FIG. 7 shows an evaluation of ultrasound calibration using an eight-point phantom as illustrated in FIG. 7a; FIG. 7b shows an LUS B-mode scan of pins on the phantom; and FIG. 7c shows 3D positions of eight pins obtained from tracked LUS scans (depicted in yellow), while ground truth positions of the eight pins are also shown (depicted in green).

For the experiment, the eight pins on the phantom were scanned in turn using the LUS probe. The pin heads were manually segmented from the US images, and 100 frames were collected at each pin to minimise the impact of manual segmentation error. The 3D positions of the pins in the EM coordinate system were computed by multiplying the 2D pixel location by the calibration transformation and then the EM tracking transformation. The accuracy of the computed 3D positions was then assessed based on two ground truths. The first ground truth is the known geometry of the 8-pin phantom, in which the pins are arranged on a 4×2 grid, with each side being 25 mm in length. The resulting mean edge length determined in the experiment was 24.62 mm. The second ground truth is the physical positions of the eight phantom pins in the EM coordinate system, which are measured by using another EM sensor tracked by the same EM transmitter. The distance between each reconstructed pin and its ground truth position is listed in Table 1.

TABLE 1
Error measures for each reconstructed pin position

| | Pin number | | | | | | | |
|---|---|---|---|---|---|---|---|---|
| | 1 | 2 | 3 | 4 | 5 | 6 | 7 | 8 |
| RMS error | 2.89 | 3.40 | 1.28 | 0.81 | 2.35 | 1.59 | 2.20 | 2.82 |
| (mm) | | | | | | | | |

### Vessel Segmentation Error

The LUS images were acquired from a phantom made from Agar. The phantom contained tubular structures filled with water. The ground truth is the diameter of the tubular structures, which are manufactured with a diameter of 6.5 mm. One hundred and sixty images (640×480 pixels) were collected. The contours of the tubular structures were automatically segmented from the US images and fitted with ellipses, so that the short ellipse axis approximated the diameter of the tubular structures. The resulting mean (standard deviation) diameter of the segmented contours was 6.4 (0.17) mm. The average time of the image processing for one US image was 100 ms.

The above calibration is illustrated in FIG. 8, which shows the validation of vessel segmentation using the phantom. In particular, FIG. 8a shows the phantom design (the rods are removed after filling the box with Agar); FIG. 8b shows an LUS probe being swept across the surface of the phantom which is now formed from the agar. An EM sensor is attached to the LUS probe and tracked. FIGS. 8c-e show LUS images of the tubular structures at various positions and orientations. The outlines of these tubular structures are shown depicted in red; the ellipses fitted to the outlines are shown depicted in green; and the extracted ellipse centres are shown by the dots/points in the images depicted in green.

### Registration Accuracy: Phantom

The registration accuracy was assessed on the same phantom as used for the preceding section “Vessel segmentation error”; (see also FIG. 8). Using the approach discussed above, the tubular structures were automatically segmented, and the centre points of these structures were extracted and converted to EM coordinates by multiplication with the US calibration matrix and the EM tracker matrix. These reconstructed points were then rigidly registered to the centre lines of the phantom tubular structures using the ICP method.

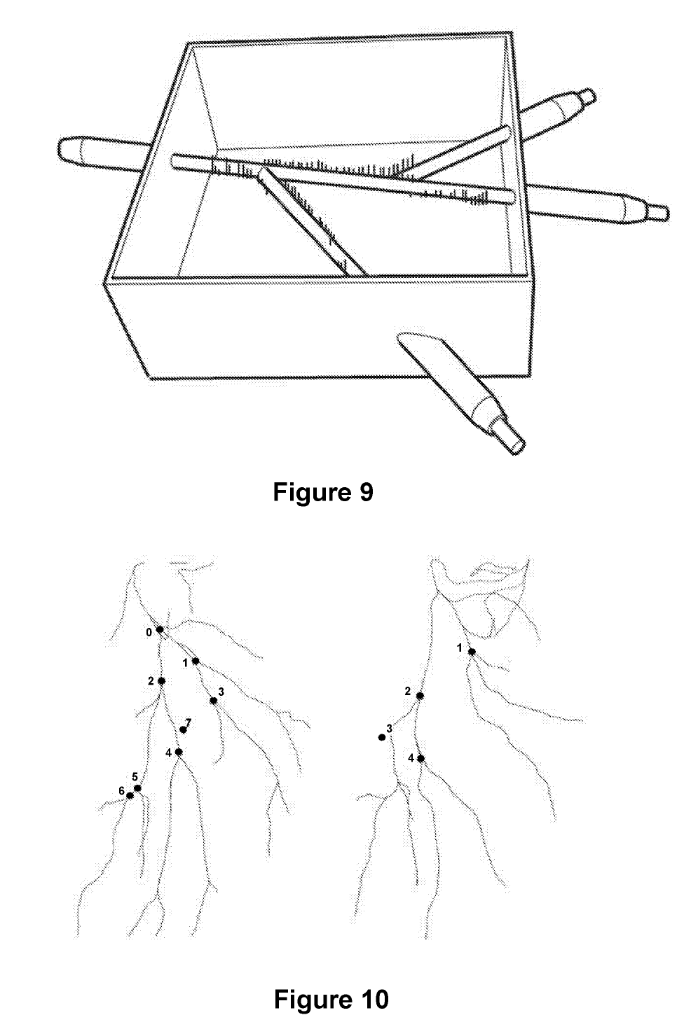

FIG. 9 shows the validation of vessel registration on the phantom of FIG. 8a. The reconstructed contours from the ultrasound data (yellow rings) were rigidly registered to the phantom using ICP. FIG. 9 illustrates in particular the registration of reconstructed points to the phantom model. The RMS residual error given by the ICP method was 0.7 mm.

### Registration Accuracy: In Vivo

The overall registration accuracy was evaluated during porcine laparoscopic liver resection using two studies of the same subject. The LUS images were acquired from the left lobe of the liver, before and after a significant repositioning of the lobe. The surgeon swept the liver surface in a steady way to make sure vessel centre points were densely sampled in the LUS images and gently so as not to cause significant deformation of the liver surface. The US imaging parameters for brightness, contrast and gain control were preset values and did not change during the scanning. About 10 LUS images per second were segmented.

In the first study, in total 370 images (640×480 pixels) were processed. In the second study, 340 images were processed. The detected vessel centres from the US images were converted into 3D data points P. Two tri-phase clinical CT scans had been obtained a week earlier, one with insufflation (12 mm Hg) and one without. Vessel centre lines were extracted using the model derived from the insufflated CT scan. The registration method described above was utilised (see also FIG. 6), in which the pre-operative centre-line model G is registered to the intra-operative data set P.

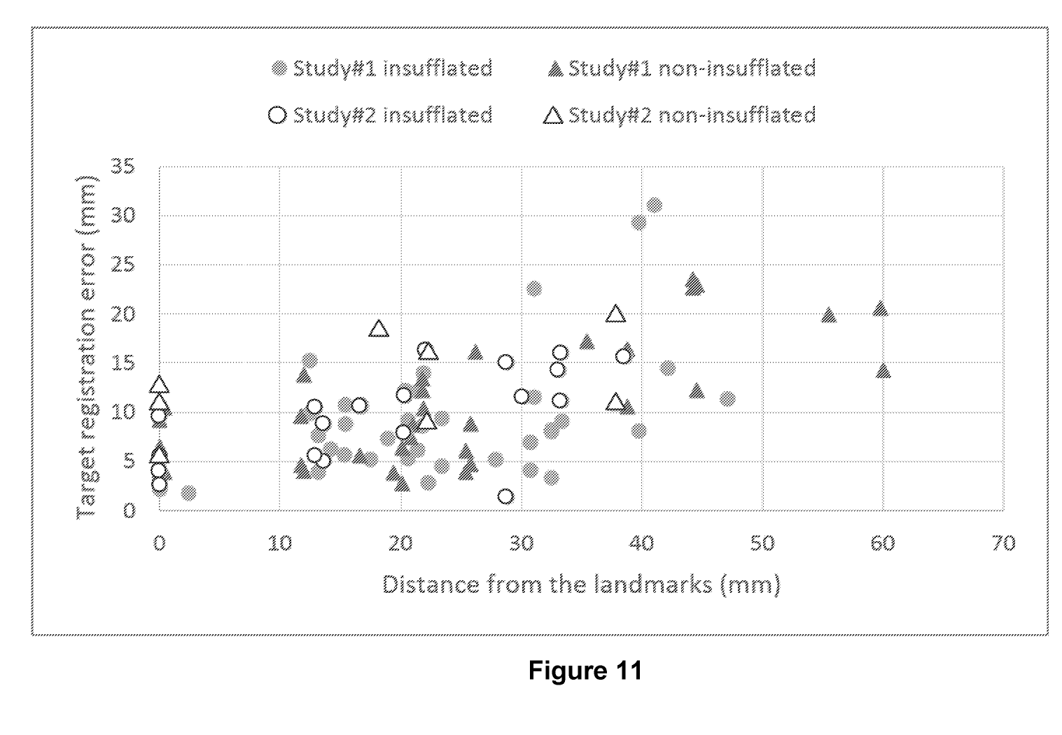

FIG. 10 depicts various hepatic vein landmark positions which were used for the image registration. In particular, FIG. 10a shows eight bifurcation landmarks on the centre-line model obtained from the pre-operative image data, which were used to measure target registration error (TRE) in a first study; FIG. 10b shows three bifurcation landmarks on the centre-line model which were used to measure TRE in the second study.

For the first study therefore, after manually identifying and labelling the eight bifurcations in both the US images and the CT data, these landmarks were then used as anatomical targets. The mean target registration error (TRE) for these anatomical targets was 3.58 mm, and the maximum TRE was 5.76 mm. For the second study, three bifurcations (i.e., numbers 1, 2, 4 in FIG. 10b) were identified for use as anatomical targets, as only the middle part of the left lobe of the liver was scanned. The mean TRE of the anatomical targets for this second study was 2.99 mm and the maximum TRE was 4.37 mm.

### Navigation Accuracy: In Vivo

To evaluate the navigation accuracy, the surgeon scanned another LUS image sequence for each of the first and second studies (giving four US data sets in total), again using minimal force on the LUS probe to avoid deformation. Using the same bifurcation landmarks as in previous registration experiment (section “Registration accuracy: in vivo”), the corresponding landmarks in the LUS images were manually identified. For the first study, the mean TRE was 4.48 mm and the maximum TRE was 7.18 mm. For the second study, the mean TRE was 3.71 mm and the maximum TRE was 4.40 mm.

### Comparison of Insufflated Versus Non-Insufflated Models

In the above sections “Registration accuracy: in vivo” and “Navigation accuracy: in vivo”, the insufflated CT model was used to evaluate the registration and navigation accuracy. In clinical practice, the patient would be scanned without insufflation, so in this section vessel centre lines derived from both insufflated and non-insufflated CT data were used. From the first study, landmarks 1, 2, 4, 5 (see FIG. 10a) were manually identified and labelled in both the US images and the CT data. From the second study, landmarks 1, 2, 4 (see FIG. 10b) were used. A registration was then performed for each landmark to register the CT data to the US using the manual registration method (a landmark and two vectors, illustrated above in FIG. 6).

For each registration, the TRE was evaluated as in section “Registration accuracy: in vivo” using the eight bifurcations for the first study and the three bifurcations for the second study. The measures of TRE are presented graphically in FIG. 11, which depicts an evaluation of registration accuracy with locally rigid registration. The errors are shown as a function of distance from the landmark used to perform the registration. Within 35 mm distance from the reference points, 76% landmarks have a TRE smaller or equal to 10 mm with the insufflated CT model; 72% for the non-insufflated CT model.

Similarly the navigation error is measured on the second LUS sequence for each study for each locally rigid registration. The measures of navigation error are illustrated in FIG. 12, which shows an evaluation of navigation accuracy with locally rigid registration. The errors are shown as a function of distance from the reference landmarks. Within 35 mm distance from the reference points, 74% landmarks have TRE smaller or equal to 10 mm with the insufflated CT model; 71% for the non-insufflated CT model.

### Discussion

A practical laparoscopic image guidance system is described and evaluated herein, which is based on a fast and accurate vessel centre-point reconstruction coupled with a locally rigid registration to a pre-operative model (or image data) using vascular features visible in LUS images.

In the above section “Ultrasound calibration error”, the accuracy of the invariant point calibration method was investigated. The mean edge length between pins in the 8-pin phantom was 24.62 mm compared with a manufactured edge length of 25 mm. Table 1 shows the reconstructed physical position errors between 0.81 and 3.40 mm, and an average of 2.17 mm, and this includes errors in measuring the gold standard itself. It is concluded that the accuracy of the approach described herein is comparable to other methods such as [17], which are typically more complex in approach. The segmentation accuracy on a plastic phantom was also investigated (see the section “Vessel segmentation error”). The phantom was constructed via 3D printing a computer-aided design (CAD) model and had known geometry with a tolerance of 0.1 mm. The reconstructed size of the internal diameter of the tubes using the approach described herein was 6.4 mm compared with the diameter in the CAD model of 6.5 mm and was deemed within tolerance. Furthermore, in the section “Registration accuracy: phantom” it is seen that the ICP-based registration of the point cloud, resulting from the US segmentation to the CAD model itself, gave an RMS error of 0.7 mm.

In the above section “Registration accuracy: in vivo”, the registration accuracy is evaluated in two in vivo studies. The mean TRE from these two studies was 3.58 and 2.99 mm, measured at eight and three identifiable landmarks, respectively. This represents a best case scenario for rigid registration, as an insufflated CT model and a large region of interest (left temporal lobe) were used.

The above assessment of accuracy does not allow for movement due to respiration and cardiac pulsatile motion. Controlled breathing means that most of the time is spent near maximum exhale. For data collected over (say) 40 seconds, corresponding to several breathing cycles, and using ICP-based methods over a large region of interest, it is believed that the data will be somewhat noisy, but the registration will average over the noise. Other possibilities are to utilise breath-holding techniques, faster software or a footswitch synchronised to the breathing, especially in conjunction with manual landmark-based registration. During the cardiac cycle, vessels pulsate and change size; however the approach described herein mitigates against this problem by using vessel centre lines, which should be more reliable and consistent than vessel external contours.

From the initial registration, a second test data set was used to evaluate navigation accuracy. This second test incorporates the error due to registration, additional nonlinear EM tracking errors and errors due to further liver deformation via the US probe. Comparing the TRE errors of the corresponding data set in the above sections “Registration accuracy: in vivo” and “Navigation accuracy: in vivo”, the navigation accuracy is only slightly worse than the registration accuracy, given that the surgeon performed the US scans in a consistent way. This suggests the EM tracking error is not a major problem.

In clinical practice, the patient will not be CT scanned while insufflated. The pre-operative, non-insufflated CT will have a significantly different shape to that seen during surgery, so registration of both insufflated and non-insufflated CT has been compared herein. However, it was somewhat difficult to identify corresponding landmarks in both CT scans, so rather than having eight landmarks in study 1, only landmarks labelled as 1, 2, 4 and 5 in FIG. 10a were identified consistently in both insufflated and non-insufflated CT models.

If a large region of interest is scanned using the US probe, the ICP-based registration to non-insufflated CT models may be less reliable, due to the significantly different shape. If a small region of interest is scanned, then the smaller the structure present in that region of interest, and the more likely the structure is to be featureless, e.g., more closely resembling a line. Thus in order to directly compare insufflated with non-insufflated registration, the manual landmark-based method (section “Registration”) was used around individual bifurcations, so as to be consistent across the two studies. Comparing FIGS. 11 and 12, it can be seen that there are similar errors when using non-insufflated or insufflated errors, but an acceptable level (<5 mm) is achievable only in the regions that are relatively near to a registration point. Interestingly, the navigation errors are not dissimilar for non-insufflated or insufflated cases: locally rigid registrations were tested on both insufflated and non-insufflated CT models and gave respective mean (standard deviation) errors of 4.23 (2.18) mm and 6.57 (3.41) mm, when measured at target landmarks located within 10 mm of a landmark used for the registration.

When measured within 35 mm of the reference points, over 70% of the target landmarks have errors smaller than or equal to 10 mm for both CT models (insufflated and non-insufflated). FIGS. 11 and 12 confirm that if TREs are assessed away from the reference points, then errors do indeed increase.

By way of comparison of the above errors with to existing finite element methods that attempt to compensate for tissue deformation (rather than using a rigid registration as described herein), Suwelack et al. [23] measured errors of 5.05 mm and 8.7 mm on a liver phantom, Haouchine et al. [13] measured registration accuracy at two points as 2.2 and 5.3 in an ex vivo trial, and Bano et al. [3] measured 4 mm error at the liver surface but 10 mm error at structures internal to the liver. Although deformable models based on understanding the biomechanics of tissue deformation are developing rapidly [3, 13, 23], there remain significant issues of validation in a surgical environment. It is anticipated that it will be a long time before surgeons have sufficient faith in a deforming model alone to guide surgical decisions during resection itself. However, the locally rigid registration system described herein is practical and could relatively easily be automated with minimal user intervention. A further possibility is that such locally rigid registrations could be used to drive and validate a deformable model.

In the implementations described above, vessel centre-lines are extracted from the pre-operative CT or MR image data. However, in other embodiments, other data may be extracted from the pre-operative data and used in the registration process, such as vessel contours as opposed to centre lines are extracted. As a further alternative, the dimensions of the vessels may also be extracted; in this case, the vessel sizing can (for example) be used to assist in identifying landmarks within the image data for use in registration as described above. Similarly, while the above implementation involves deriving vessel centre-points from the 2D US images, in other implementations, other parameters, such as vessel contours, may be derived (instead of or in addition to the vessel centre-points).

In the implementations described above, bifurcation points are primarily utilised as anatomical landmarks. However, it should be understood that other landmarks may be used instead—for instance, locations where a given vessel enters or exits a particular organ, or has a particular looped configuration, etc. Moreover, although the bifurcation landmarks are manually located in the above processing, the automatic identification of suitable landmarks may also be performed in at least one of the images or data sets (i.e., pre-operative or intra-operative).

Furthermore, it is also described above that there is an initial registration based upon the identified landmarks in the vessel centre points and vessel centre lines before an ICP algorithm is used to refine the registration. In some implementations, a single algorithm may be used for the alignment, or alternatively, the CT/MR image data may be manipulated by the clinician based upon a visual assessment to provide (or at least estimate) the registration, which may then be confirmed by suitable processing.

As shown by the experimental the above results, the method described herein is sufficiently accurate to provide a useful form of image registration, although further validation, e.g. using animal models, is desirable (and would generally be required prior to clinical adoption). In some implementations, a simple user interface may be provided that, based on a sufficiently close initial estimate, allows the liver (or other soft deforming organ) to be scanned round the target lesion and nearby vessel bifurcations. With this approach, it may be possible to obtain registration errors of the order of 4-6 mm with no deformable modelling. The method is both practical and provides guidance to the surgical target. It also implicitly includes information on the location of nearby vasculature structures, which are the same structures a surgeon needs to be aware of when undertaking laparoscopic resection. This may also provide advantages over open surgery and haptics, where the surgeon generally remains blind to the precise location of these structures.

The apparatus described herein may perform a number of software-controlled operations. In such cases, the software may run at least in part on special-purpose hardware (e.g. GPUs) or on a conventional computer system having a generic processors. The software may be loaded into such hardware, for example, by a wireless or wired communications link, or may be loaded by some other mechanism—e.g. from a hard disk drive, or a flash memory device.

The skilled person will appreciate that various embodiments have been described herein by way of example, and that different features from different embodiments can be combined as appropriate. Accordingly, the scope of the presently claimed invention is to be defined by the appended claims and their equivalents.

### Acknowledgments

This publication presents independent research funded by the Health Innovation Challenge Fund (HICF-T4-317), a parallel funding partnership between the Wellcome Trust and the Department of Health. The views expressed in this publication are those of the author(s) and not necessarily those of the Wellcome Trust or the Department of Health. DB and DJH received funding from EPSRC EP/F025750/1. SO and DJH receive funding from EPSRC EP/H046410/1 and the National Institute for Health Research (NIHR) University College London Hospitals Biomedical Research Centre (BRC) High Impact Initiative. We would like to thank NVidia Corporation for the donation of the Quadro K5000 and SDI capture cards used in this research.

### REFERENCES

[1] Anderegg S, Peterhans M, Weber S (2010) “Ultrasound segmentation in navigated liver surgery”, http://www.cascination.com/information/publications/
[2] Aylward S R, Jomier J, Guyon J P, Weeks S (2002) “Intra-operative 3D ultrasound augmentation”. In: Proceedings, 2002 IEEE international symposium on biomedical imaging, pp 421-424. IEEE, doi:10.1109/ISB1.2002.1029284
[3] Bano J, Nicolau S, Hostettler A, Doignon C, Marescaux J, Soler L (2013) “Registration of preoperative liver model for laparoscopic surgery from intraoperative 3d acquisition”. In: Liao H, Linte C, Masamune K, Peters T, Zheng G (eds) Augmented reality environments for medical imaging and computer-assisted interventions. Lecture notes in computer science, vol 8090. Springer, Berlin, pp 201-210. doi:10.1007/978-3-642-40843-4_22
[4] Barratt D C, Davies A H, Hughes A D, Thom S A, Humphries K N (2001) “Accuracy of an electromagnetic three-dimensional ultrasound system for carotid artery imaging”. Ultrasound Med Biol 27(10):1421-1425
[5] Best P J, McKay N D (1992) “Method for registration of 3-D shapes”. In: Robotics-D L tentative. International Society for Optics and Photonics, pp 586-606. doi:10.1117/12.57955
[6] Clarkson M, Zombori G, Thompson S, Totz J, Song Y, Espak M, Johnsen S, Hawkes D, Ourselin S (2015) “The NifTK software platform for image-guided interventions: platform overview and NiftyLink messaging”. Int J Comput Assist Radiol Surg 10(3):301-316. doi:10.1007/s11548-014-1124-7
[7] Croome K P, Yamashita M H (2010) “Laparoscopic vs open hepatic resection for benign and malignant tumors: an updated metaanalysis”. Arch Surg 145(11):1109-1118. doi:10.1001/archsurg.2010.227
[8] Dagon B, Baur C, Bettschart V (2008) “Real-time update of 3D deformable models for computer aided liver surgery”. In: 19th international conference on pattern recognition (ICPR 2008), pp. 1-4. IEEE. doi:10.1109/ICPR.2008.4761741
[9] Feuerstein M, Reichl T, Vogel J, Traub J, Navab N(2009) “Magnetooptical tracking of flexible laparoscopic ultrasound: model-based online detection and correction of magnetic tracking errors”. IEEE Trans Med Imaging 28(6):951-967. doi:10.1109/TMI.2008.2008954
[10] Frangi A, Niessen W, Vincken K, Viergever M (1998) “Multiscale vessel enhancement filtering”. In: Wells W, Colchester A, Delp S (eds) Medical image computing and computer-assisted interventation MICCAI98. Lecture notes in computer science, vol 1496. Springer, Berlin, pp 130-137. doi:10.1007/BFb0056195
[11] Franz A, Haidegger T, Birkfellner W, Cleary K, Peters T, Maier-Hein L (2014) “Electromagnetic tracking in medicine 2014: a review of technology, validation, and applications”. IEEE TransMed Imaging 33(8):1702-1725. doi:10.1109/TMI.2014.2321777
[12] Guerrero J, Salcudean S, McEwen J, Masri B, Nicolaou S (2007) “Real-time vessel segmentation and tracking for ultrasound imaging applications”. IEEE Trans Med Imaging 26(8):1079-1090. doi:10.1109/TMI.2007.899180
[13] Haouchine N, Dequidt J, Peterlikl, Kerrien E, Berger M O, Cotin S (2013) “Image-guided simulation of heterogeneous tissue deformation for augmented reality during hepatic surgery”. In: 2013 IEEE international symposium on mixed and augmented reality (ISMAR), pp 199-208. doi:10.1109/ISMAR.2013.6671780
[14] Kingham T P, Jayaraman S, Clements L W, Scherer M A, Stefansic J D, Jarnagin W R (2013) “Evolution of image-guided liver surgery: transition from open to laparoscopic procedures”. J Gastrointest Surg 17(7):1274-1282. doi:10.1007/s11605-013-2214-5
[15] Lange T, Eulenstein S, Hünerbein M, Schlag P M (2003) “Vessel based non-rigid registration of MR/CT and 3D ultrasound for navigation in liver surgery”. Comput Aided Surg 8(5):228-240. doi:10.3109/10929080309146058
[16] Lange T, Papenberg N, Heldmann S, Modersitzki J, Fischer B, Lamecker H, Schlag P M (2009) “3D ultrasound-CT registration of the liver using combined landmark-intensity information”. Int J Comput Assist Radiol Surg 4(1):79-88. doi:10.1007/s11548-008-0270-1
[17] Mercier L, Lang T, Lindseth F, Collins L D (2005) “A review of calibration techniques for freehand 3-d ultrasound systems”. Ultrasound Med Biol 31(2):143-165. doi:10.1016/j.ultrasmedbio.2004.11.001
[18] Nakada K, Nakamoto M, SatoY, KonishiK, Hashizume M, Tamura S (2003) “A rapid method for magnetic tracker calibration using a magneto-optic hybrid tracker”. In: Ellis R, Peters T (eds) Medical image computing and computer-assisted intervention-MICCAI 2003. Lecture notes in computer science, vol 2879. Springer, Berlin, pp 285-293. doi:10.1007/978-3-540-39903-2_36
[19] Nicolau S, Soler L, Mutter D, Marescaux J (2011) “Augmented reality in laparoscopic surgical oncology”. Surg Oncol 20(3):189-201. doi:10.1016/j.suronc.2011.07.002
[20] Noble J, Boukerroui D (2006) “Ultrasound image segmentation: a survey”. IEEE Trans Med Imaging 25(8):987-1010. doi:10.1109/TMI.2006.877092
[21] Penney G P, Blackall J M, Hamady M, Sabharwal T, Adam A, Hawkes D J (2004) “Registration of freehand 3Dultrasound and magnetic resonance liver images”. Med Image Anal 8(1):81-91. doi:10.1016/j.media.2003.07.003
[22] Schneider C, Guerrero J, Nguan C, Rohling R, Salcudean S (2011) “Intra-operative pick-up ultrasound for robot assisted surgery with vessel extraction and registration: a feasibility study”. In: Taylor R, Yang G Z (eds) Information processing in computer-assisted interventions. Lecture notes in computer science, vol 6689. Springer, Berlin, pp 122-132. doi:10.1007/978-3-642-21504-9_12
[23] Suwelack S, Rhl S, Bodenstedt S, Reichard D, Dillmann R, dos Santos T, Maier-Hein L, Wagner M, Wnscher J, Kenngott H, Miler B P, Speidel S (2014) “Physics-based shape matching for intraoperative image guidance”. Med Phys 41(11):111901. doi:10.1118/1.4896021
[24] Thompson S, Totz J, Song Y, Stoyanov D, Ourselin S, Hawkes D J, Clarkson M J (2015) “Accuracy validation of an imageguided laparoscopy system for liver resection”. In: Proceedings of SPIE medical imaging
[25] Totz J, Thompson S, Stoyanov D, Gurusamy K, Davidson B, Hawkes D J, Clarkson M J (2014) Fast semi-dense surface reconstruction from stereoscopic video in laparoscopic surgery. In: Stoyanov D, Collins D, Sakuma I, Abolmaesumi P, Jannin P (eds) Information processing in computer-assisted interventions. Lecture notes in computer science, vol 8498. Springer, pp 206-215. doi:10.1007/978-3-319-07521-1_22
[26] Wein W, Brunke S, Khamene A, Callstrom M R, Navab N (2008) “Automatic C T-ultrasound registration for diagnostic imaging and image-guided intervention”. Med Image Anal 12(5):577-585. doi:10.1016/j.media.2008.06.006
[27] Wein W, Ladikos A, Fuerst B, Shah A, Sharma K, Navab N (2013) “Global registration of ultrasound to mri using the LC2 metric for enabling neurosurgical guidance”. In: Mori K, Sakuma I, Sato Y, Barillot C, Navab N (eds) Medical image computing and computer assisted intervention-MICCAI 2013. Springer, Berlin, Heidelberg, pp 34-41. doi:10.1007/978-3-642-40811-3_5

What is claimed is:

1. A method for registering pre-operative three dimensional (3-D) image data of a deformable organ comprising vessels with multiple intra-operative two-dimensional (2-D) ultrasound images of the deformable organ acquired by a laparoscopic ultrasound probe during a laparoscopic procedure, the method comprising:
generating a 3-D vessel graph from the 3-D pre-operative image data;
using the multiple 2-D ultrasound images to identify 3-D vessel locations in the deformable organ;
determining a rigid registration between the 3-D vessel graph from the 3-D pre-operative image data and the identified 3-D vessel locations in the deformable organ; and
applying said rigid registration to align the pre-operative three dimensional (3-D) image data with the two-dimensional (2-D) ultrasound images, wherein the rigid registration is locally valid in the region of the deformable organ of interest for the laparoscopic procedure.

2. The method of claim 1, wherein pre-operative three dimensional (3-D) image data comprises magnetic resonance (MR) or computed tomography (CT) image data.

3. The method of claim 1, wherein the multiple intra-operative two-dimensional (2-D) ultrasound images comprise 2D ultrasound slices at different orientations and positions through the region of the deformable organ of interest for the laparoscopic procedure.

4. The method of claim 1, wherein the laparoscopic ultrasound probe includes a tracker to provide tracking information for the probe that allows the 2D ultrasound slices at different orientations and positions to be mapped into a consistent 3-D space.

5. The method of claim 1, wherein generating a 3-D vessel graph from the 3-D pre-operative image data comprises:
segmenting the 3-D pre-operative image data into anatomical features including the vessels; and
identifying the centre-lines of the segmented vessels to generate the 3-D vessel graph.

6. The method of claim 1, wherein using the multiple 2-D ultrasound images to identify 3-D vessel locations in the deformable organ comprises:
identifying the locations of vessels within individual 2-D ultrasound images; and
converting the identified locations of vessels within an individual 2-D ultrasound image into corresponding 3-D locations of vessels using tracking information for the laparoscopic ultrasound probe.

7. The method of claim 6, wherein the locations of vessels within individual 2-D ultrasound images comprise vessel centre-points.

8. The method of claim 6, wherein identifying the locations of vessels within an individual 2-D ultrasound images comprises:
applying a vessel enhancement filter to the individual ultrasound image;
thresholding the filtered image; and
fitting ellipses to the thresholded image, whereby a fitted ellipse corresponds to a cross-section through a vessel in the individual ultrasound image.

9. The method of claim 8, further comprising:
creating a Dip image from the individual ultrasound image; and
apply the Dip image as a mask to the thresholded image.

10. The method of claim 8, further comprises excluding, as a location of vessel, a fitted ellipse having a high eccentricity.

11. The method of claim 1, wherein determining the rigid registration between the 3-D vessel graph and the identified 3-D vessel locations in the deformable organ includes determining an initial alignment based on two or more corresponding anatomical landmarks in the 3-D vessel graph from the pre-operative image data and the identified 3-D vessel locations from the intra-operative ultrasound images.

12. The method of claim 11, wherein the initial alignment is performed by manually identifying the corresponding anatomical landmarks.

13. The method of claim 11, wherein the anatomical landmarks comprise vessel bifurcations.

14. The method of claim 1, wherein determining the rigid registration includes determining an alignment between the 3-D vessel graph from the pre-operative image data and points representing the identified 3-D vessel locations from the intra-operative ultrasound images using an iterative closest points algorithm.

15. The method of claim 1, wherein the identified 3-D vessel locations comprise a cloud of points in 3D space, each point representing the centre-point of a vessel, wherein the vessel graph comprises the centre-lines of the vessels identified in the pre-operative image data, and wherein the rigid registration is determined between the vessel graph of centre-lines and the cloud of points.

16. The method of claim 1, further comprising providing a real-time, intra-operative, display of the pre-operative three dimensional (3-D) image data registered with the two-dimensional (2-D) ultrasound images.

17. The method of claim 16, wherein the laparoscopic ultrasound probe includes a video camera, and the method further comprising displaying a video image from the video camera in alignment with the three dimensional (3-D) image data and the two-dimensional (2-D) ultrasound images.

18. The method of claim 1, wherein the deformable organ is the liver.

19. A non-transitory computer-readable medium comprising program instructions that when executed on a computer cause the computer to perform a
method for registering pre-operative three dimensional (3-D) image data of a deformable organ comprising vessels with multiple intra-operative two-dimensional (2-D) ultrasound images of the deformable organ acquired by a laparoscopic ultrasound probe during a laparoscopic procedure, the method comprising:
generating a 3-D vessel graph from the 3-D pre-operative image data;
using the multiple 2-D ultrasound images to identify 3-D vessel locations in the deformable organ;
determining a rigid registration between the 3-D vessel graph from the 3-D pre-operative image data and the identified 3-D vessel locations in the deformable organ; and
applying said rigid registration to align the pre-operative three dimensional (3-D) image data with the two-dimensional (2-D) ultrasound images, wherein the rigid registration is locally valid in the region of the deformable organ of interest for the laparoscopic procedure.

20. Apparatus for registering pre-operative three dimensional (3-D) image data of a deformable organ comprising vessels with multiple intra-operative two-dimensional (2-D) ultrasound images of the deformable organ acquired by a laparoscopic ultrasound probe during a laparoscopic procedure, the apparatus being configured to:
generate a 3-D vessel graph from the 3-D pre-operative image data;
use the multiple 2-D ultrasound images to identify 3-D vessel locations in the deformable organ;
determine a rigid registration between the 3-D vessel graph from the 3-D pre-operative image data and the identified 3-D vessel locations in the deformable organ; and
apply said rigid registration to align the pre-operative three dimensional (3-D) image data with the two-dimensional (2-D) ultrasound images, wherein the rigid registration is locally valid in the region of the deformable organ of interest for the laparoscopic procedure.

21. (canceled)

22. (canceled)
